(19) United States
(12) Patent Application Publication
(10) Pub. No.: US 2016/0102140 A1
(43) Pub. Date: Apr. 14, 2016

(54) METHODS AND COMPOSITIONS FOR TREATING BRAIN DISEASES

(71) Applicant: ARIZONA BOARD OF REGENTS ON BEHALF OF ARIZONA STATE UNIVERSITY, Scottsdale, AZ (US)

(72) Inventor: Michael SIERKS, Ft. McDowell, AZ (US)

(73) Assignee: ARIZONA BOARD OF REGENTS ON BEHALF OF ARIZONA STATE UNIVERSITY, Scottsdale, AZ (US)

(21) Appl. No.: 14/894,285

(22) PCT Filed: May 30, 2014
(86) PCT No.: PCT/US14/40342

§ 371 Date: Nov. 25, 2015

Related U.S. Application Data
(60) Provisional application No. 61/829,120, filed on May 30, 2013.

(51) Int. Cl. C07K 16/18; C12N 7/00

(52) U.S. Cl. CPC C07K 16/18; C12N 7/00; C12N 2740/15043; C07K 2319/10; C07K 2317/569; C07K 2317/76; A61K 2039/505

(57) ABSTRACT

The present invention provides methods of delivering a protein to the brain of a mammal, comprising administering to the mammal a therapeutic fusion protein comprising a homeodomain peptide operably linked to a therapeutic agent.

### RELATED APPLICATION

This application claims the benefit under 35 U.S.C. §119(e) of U.S. Provisional Application No. 61/829,120, filed May 30, 2013, the entire contents of which is hereby incorporated by reference.

### BACKGROUND

Treatment of diseases of the central nervous system, e.g., inherited genetic diseases of the brain, remains an intractable problem. Examples of such are the lysosomal storage diseases. Collectively, the incidence of lysosomal storage diseases (LSD) is 1 in 10,000 births world wide, and in 65% of cases, there is significant central nervous system (CNS) involvement. Proteins deficient in these disorders, when delivered intravenously, do not cross the blood-brain barrier, or, when delivered directly to the brain, are not widely distributed. Thus, therapies for the CNS deficits need to be developed.

Gene transfer is now widely recognized as a powerful tool for analysis of biological events and disease processes at both the cellular and molecular level. More recently, the application of gene therapy for the treatment of human diseases, either inherited (e.g., ADA deficiency) or acquired (e.g., cancer or infectious disease), has received considerable attention. With the advent of improved gene transfer techniques and the identification of an ever expanding library of defective gene-related diseases, gene therapy has rapidly evolved from a treatment theory to a practical reality.

### SUMMARY

In certain embodiments, the present invention provides a method of delivering a protein to the brain of a mammal, comprising administering to the mammal a therapeutic fusion protein comprising a homeodomain peptide operably linked to a therapeutic agent, wherein the homeodomain peptide tag is 15-35 amino acids in length, has at least 80% identity to SEQ ID NO:5 or SEQ ID NO:6, has cellular penetration and secretion functions, and facilitates blood-brain barrier transport of a therapeutic agent in the mammal.

In certain embodiments, the present invention provides a method of delivering a protein to the brain of a mammal, comprising administering to the mammal a viral vector comprising a nucleic acid sequence encoding a homeodomain peptide tag operably linked to a therapeutic agent. In certain embodiments, the viral vector is a lentiviral vector.

In certain embodiments, the present invention provides a fusion protein comprising a homeodomain peptide tag that facilitates transport of a therapeutic antibody fragment across a blood-brain barrier operably linked to a therapeutic antibody fragment, wherein the homeodomain peptide tag is 15-35 amino acids in length, has at least 80% identity to SEQ ID NO:5 or SEQ ID NO:6, and has cellular penetration and secretion functions.

In certain embodiments, the present invention provides a viral vector comprising a nucleic acid sequence encoding a homeodomain peptide tag operably linked to a therapeutic agent. In certain embodiments, the viral vector is a lentiviral vector.

In certain embodiments, the homeodomain peptide tag has at least 95% identity to SEQ ID NO:5 or SEQ ID NO:6. In certain embodiments, the homeodomain peptide tag has 100% identity to SEQ ID NO:5 or SEQ ID NO:6. In certain embodiments, the therapeutic agent is an antibody fragment. In certain embodiments, the antibody fragment is D5 or 10H. In certain embodiments, the therapeutic fusion protein further comprises a linker positioned between the homeodomain peptide tag and the therapeutic agent. In certain embodiments, the mammal is human.

In certain embodiments, the present invention provides a cell comprising the viral vector described above.

In certain embodiments, the present invention provides a cell transduced by the viral vector described above. In certain embodiments, the cell is a mammalian cell. In certain embodiments, the cell is a human cell.

In certain embodiments, the present invention provides a viral vector described above, for use in medical treatment or diagnosis.

In certain embodiments, the present invention provides a cell as described above for use in medical treatment or diagnosis.

### BRIEF DESCRIPTION OF THE DRAWINGS

FIG. 1. A-syn levels in control and transgenic mouse neurons treated with lentivirus expressing D5. Significant intracellular production of the D5 nanobody can be observed in both transfected control and a-syn neurons receiving the LV-D5 treatment. Substantial a-syn accumulation is observed in the a-syn cells receiving the control lentivirus, but decreased a-syn is observed in the a-syn cells transfected with D5.

FIG. 2. A-syn levels in control and transgenic mouse neurons treated with lentivirus expressing D5 tagged with a secretion signal. Only low levels of D5 nanobody can be observed in both transfected control and a-syn neurons receiving the LV-cd5-D5 treatment compared to levels of untagged D5 observed in FIG. 1. Substantial a-syn accumulation is again observed in the a-syn cells receiving the control lentivirus, but substantially reduced a-syn levels are observed in the a-syn cells transfected with cd5-D5.

FIG. 3. A-syn levels in control and transgenic brain tissue treated with lentivirus expressing D5. Upper row shows presence of D5 in the brain tissue of mice (wt and tg) treated with the cd5-D5 nanobody. Middle row shows levels of a-syn in the wt and tg mouse brain tissue. Only low levels of a-syn are observed in the non-tg brain tissue samples, the highest a-syn levels are observed in the tg mouse sample with control LV, and over 50% decrease in a-syn staining is observed with the tg sample treated with cd5-D5. Lower row shows a-syn staining with increased magnification. Substantial aggregates are observed with the tg mouse brain receiving the control LV, and greatly reduced aggregates in the tg mouse brain receiving the CD5-D5 antibody.

FIG. 4. Neuronal health as measured by NeuN and GFAP in wt and tg mouse models. Top row shows levels of NeuN staining in treated and control wt mouse brain tissue and treated and control tg brain tissue. NeuN levels are decreased in the a-syn tg tissue, but rescued in the tg tissue treated with cd5-D5. Bottow row shows levels of GFAP staining in treated and control wt and tg mouse brain tissue. GFAP levels are elevated in the control treated a-syn tg mouse tissue, but levels are decreased to wt levels with the cd5-D5 treated a-syn tg sample.

FIG. 5. Adhesive removal test. Plot for reaction in wt (dark blue) and tg (light blue) mice treated with control, D5 and 10H.

FIG. 6. Adhesive removal test. Plot for success in wt (dark blue) and tg (light blue) mice treated with control, D5 and 10H.

FIG. 7. Adhesive removal test. Plot for Time in wt (dark blue) and tg (light blue) mice treated with control, D5 and 10H.

FIG. 8. Adhesive removal test. Plot for Removal in wt (dark blue) and tg (light blue) mice treated with control, D5 and 10H.

FIG. 9. Grip strength test. Plot of grip strength in wt (dark blue) and tg (light blue) mice treated with control, D5 and 10H.

FIG. 10. Hanging wire test. Plot of latency to fall from hanging wire in wt (dark blue) and tg (light blue) mice treated with control, D5 and 10H.

FIG. 11. Total a-synuclein levels in brain tissue of wt (NTG) and tg mice treated with bobi or with D5. Total a-syn levels are essentially the same in the Bobi and D5 treated mice.

FIG. 12. Proteinase K resistant a-syn inclusions in the substantia nigra of tg mice treated with D5 and 10H. Levels decreased the most in the D5 treated mice.

FIG. 13. Synuclein axonal dystrophy in basal ganglia of tg mice treated with D5 and 10H. Levels decreased the most in the D5 treated mice.

FIG. 14. Synuclein neuropil levels in the hippocampus of tg mice treated with D5 and 10H. Levels decreased the most in the D5 treated mice.

FIG. 15. Synuclein neuropil levels in the fronto-parietal cortex of tg mice treated with D5 and 10H. Levels decreased the most in the D5 treated mice.

FIG. 16. Total Synuclein positive cells in the fronto-parietal cortex of tg mice treated with D5 and 10H. Levels decreased the most in the D5 treated mice.

FIG. 17. Presence of D5 reactive oligomeric a-syn in brain tissue of tg and D5 and 10H treated mice measured by immunohistochemistry. Levels in D5 and 10H mice decreased to wild type levels. * Denotes p<0.05; **p<0.001.

FIG. 18. Presence of 10H reactive oligomeric a-syn in brain tissue of tg and D5 and 10H treated mice measured by immunohistochemistry. Levels in D5 and 10H mice decreased to wild type levels. * Denotes p<0.05; **p<0.001.

FIG. 19. Presence of D5 reactive oligomeric a-syn in cortex of brain tissue of tg and D5 and 10H treated mice measured by immunohistochemistry. Levels in D5 and 10H mice decreased to wild type levels. * Denotes p<0.05; **p<0.001.

FIG. 20. Presence of D5 reactive oligomeric a-syn in hippocampus of brain tissue of tg and D5 and 10H treated mice measured by immunohistochemistry. Levels in D5 and 10H mice decreased to wild type levels. * Denotes p<0.05; **p<0.001.

FIG. 21. Presence of 10H reactive oligomeric a-syn in cortex of brain tissue of tg and D5 and 10H treated mice measured by immunohistochemistry. Levels in D5 and 10H mice decreased to wild type levels. * Denotes p<0.05; **p<0.001.

FIG. 22. Presence of 10H reactive oligomeric a-syn in hippocampus of brain tissue of tg and D5 and 10H treated mice measured by immunohistochemistry. Levels in D5 and 10H mice decreased to wild type levels. * Denotes p<0.05; **p<0.001

FIG. 23. Levels of D5 reactive a-syn in homogenized mouse brain tissue as determined by ELISA. D5 treated mice show decreased D5 reactive oligomeric a-syn compared to both the control tg and control wt mice.

FIG. 24. Levels of 10H reactive a-syn in homogenized mouse brain tissue as determined by ELISA. D5 treated mice show decreased D5 reactive oligomeric a-syn compared to both the control tg and control wt mice.

FIG. 25. Levels of oligomeric beta-amyloid in homogenized mouse brain tissue as determined by ELISA. D5 treated mice show decreased oligomeric beta-amyloid compared to both the control tg and control wt mice. Oligomeric beta-amyloid was detected by reactivity with the anti-oligomeric beta-amyloid nanobody A4.

FIG. 26. Treatment with D5 and 10H helps restore neurons in the CA3 region of tg mice. Mice treated with either 10H or D5 show increased cell concentrations in the hippocampus in the tg mice compared to the control treated mice.

FIG. 27. NeuN staining in the hippocampus. Treatment with D5 and 10H helps restore neurons in the hippocampus of tg mice. Mice treated with either 10H or D5 show increased cell concentrations in the hippocampus in the tg mice compared to the control treated mice. Treatment with D5 shows improvement back to control values of non-transgenic mice.

### DETAILED DESCRIPTION

Targeting therapeutics to the brain has commercial applications for a variety of neurological disorders including Alzheimer's disease, Parkinson's disease, ALS, various dementias, brain tumors and brain infections. A peptide tag has been identified that facilitates transport of a therapeutic or diagnostic agent across the blood-brain barrier into the brain. The tag also facilitates transfer into and out of cells in the brain.

The homeodomain protein has the capacity to shuttle into and out of cells. The peptide region responsible for penetration and secretion functions has been identified. It has now been discovered that the secretion/penetration peptide sequence enables a tagged protein to facilitate transport across the blood brain barrier, thus serving as a method to deliver therapeutics to the brain.

The homeodomain secretion/penetration peptide tag is linked to a therapeutic molecule, such as a therapeutic antibody fragment. In certain embodiments, the peptide tag is linked to a therapeutic molecule by means of a chemical linkage. In certain embodiments, a nucleic acid encoding the peptide tag is linked to a nucleic acid encoding the therapeutic molecule, such that a fusion protein is generated. The fusion proteins are then purified and can be injected directly into the mammal. Further, in certain embodiments, nucleic acids encoding the peptide tag and the linked therapeutic molecule are constructed into a viral vector (e.g., a lentivirus), allowing it to be expressed in vivo. In certain embodiments, the lentivirus vector is injected intraperitoneally to infect liver and spleen cells. The liver and/or spleen cells then produced and excrete the tagged antibody fragments into the blood stream. The secretion/penetration peptide tag then facilitates transport across the blood brain barrier into the brain and into and out of cells in the brain. As described below, mice treated with antibody fragments tagged with the secretion/penetration peptide showed significant improvement in behavior and pathology compared to the control mice. The tagged antibodies have therapeutic value for treating neurological diseases since the can transport the tagged target into the brain from the blood stream.

Homeodomain Peptide Tag

In certain embodiments, the homeodomain secretion/penetration peptide tag (Sec-Pen) has the following nucleic acid sequence:

(SEQ ID NO: 1)
cagagcctggcccaggaactgggcctgaacgaacgacagatcaaaat
ctggttccagaaccgccgcatgaagtggaaaaaa
(SEQ ID NO: 2)
atgggacagagcctggcccaggaactgggcctgaacgaacgacagat
caaaatctggttccagaaccgccgcatgaagtggaaaaaa

In certain embodiments of the present invention, the Sec-Pen peptide tag is encoded by a nucleic acid having between 70% to 100% identity to SEQ ID NO:1 or SEQ ID NO:2.

In certain embodiments, the penetration portion (Pen) of the homeodomain secretion/penetration peptide tag (Sec-Pen) has the following nucleic acid sequence:

(SEQ ID NO: 3)
cgacagatcaaaatctggttccagaaccgccgcatgaagtggaaaaaa

In certain embodiments of the present invention, the Sec portion of the peptide tag is encoded by a nucleic acid having between 70% to 100% identity to SEQ ID NO:3.

In certain embodiments, the penetration portion (Sec) of the homeodomain secretion/penetration peptide tag (Sec-Pen) has the following nucleic acid sequence:

(SEQ ID NO: 4)
CAGAGcctggcccaggaactgggcctgaacgaa

In certain embodiments of the present invention, the Pen portion of the peptide tag is encoded by a nucleic acid having between 70% to 100% identity to SEQ ID NO:4.

In certain embodiments, the homeodomain secretion/penetration peptide tag (Sec-Pen) has the following amino acid sequence:

(SEQ ID NO: 5)
QSLAQELGLNERQIKIWFQNRRMKWKK
(SEQ ID NO: 6)
MGQSLAQELGLNERQIKIWFQNRRMKWKK

In certain embodiments of the present invention, the Sec-Pen peptide tag comprises between 70% to 100% identity to SEQ ID NO: 5 or SEQ ID NO: 6.

In certain embodiments, the penetration portion (Pen) of the homeodomain secretion/penetration peptide tag has the following amino acid sequence:

(SEQ ID NO: 7)
RQIKIWFQNRRMKWKK

In certain embodiments of the present invention, the Pen peptide tag comprises between 70% to 100% identity to SEQ ID NO:7.

In certain embodiments, the secretion portion (Sec) of the homeodomain secretion/penetration peptide tag has the following amino acid sequence:

(SEQ ID NO: 8)
QSLAQELGLNE

In certain embodiments of the present invention, the Sec peptide tag comprises between 70% to 100% identity to SEQ ID NO:8.

The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to a reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82:488 (1985); Kunkel et al., Meth. Enzymol., 154:367 (1987); U.S. Pat. No. 4,873,192; Walker and Gaastra, Techniques in Mol. Biol. (MacMillan Publishing Co. (1983), and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

Thus, the polypeptides of the invention encompass naturally occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

Therapeutic Agents

As used herein, the term “therapeutic agent” refers to any agent or material that has a beneficial effect on the mammalian recipient. Thus, “therapeutic agent” embraces both therapeutic and prophylactic molecules having a protein component.

“Treating” as used herein refers to ameliorating at least one symptom of, curing and/or preventing the development of a given disease or condition.

In certain embodiments, a heterologous nucleic acid can encode a therapeutic agent, such as a beneficial protein that replaces a missing or defective protein required by the subject into which the vector in transferred or can encode a cytotoxic polypeptide that can be directed, e.g., to cancer cells or other cells whose death would be beneficial to the subject. The heterologous nucleic acid can also encode antisense RNAs that can bind to, and thereby inactivate, mRNAs made by the subject that encode harmful proteins. In one embodiment, antisense polynucleotides can be produced from a heterologous expression cassette in an viral construct where the expression cassette contains a sequence that promotes cell-type specific expression.

Examples of heterologous nucleic acids which can be administered to a cell or subject as part of the present vector can include, but are not limited to the nucleic acids encoding therapeutic agents, such as lysosomal hydrolases; tumor necrosis factors (TNF), such as TNF-alpha; interferons, such as interferon-alpha, interferon-beta, and interferon-gamma; interleukins, such as IL-1, IL-1beta, and ILs-2 through-14; GM-CSF; adenosine deaminase; secreted factors such as growth factors; ion channels; chemotherapeutics; lysosomal proteins; anti-apoptotic gene products; proteins promoting neural survival such as glutamate receptors and growth factors; cellular growth factors, such as lymphokines; soluble CD4; Factor VIII; Factor IX; T-cell receptors; LDL receptor; ApoE; ApoC; alpha-1 antitrypsin; ornithine transcarbamylase (OTC); cystic fibrosis transmembrane receptor (CFTR); insulin; Fc receptors for antigen binding domains of antibodies, such as immunoglobulins; and antisense sequences which inhibit viral replication, such as antisense sequences which inhibit replication of hepatitis B or hepatitis non-A, non-B virus. Furthermore, the nucleic acid can encode more than one gene product, limited only by the size of nucleic acid that can be packaged.

In certain embodiments, any antibody or antibody fragment that targets alpha-synuclein of any form can be used as the therapeutic agent. In certain embodiments, any antibodies or antibody fragments that bind a target in the brain, such as antibodies targeting beta-amyloid or tau for Alzheimer's disease, and frontotemporal dementia, TDP-43 for ALS, antigens associated with traumatic brain injury and associated inflammatory responses, cancer antigens for brain tumors, virus or bacteria for brain infections can be used as the therapeutic agent. In certain embodiments, antibodies targeting antigens associated with depression or other psychiatric diseases can be used as the therapeutic agent. In certain embodiment, the therapeutic agent is an antibody, or antibody fragment. In certain embodiments, the antibody fragment is D5 or 10H.

Couplers/Linkers

Homeodomain peptide tag/therapeutic agent coupling is done either directly (e.g., a covalent bond) or using chemical linkers in accord with conventional practice. In certain embodiments, a fusion protein is generated such that the homeodomain peptide tag and the therapeutic agent form a single protein molecule.

In certain embodiments, the homeodomain peptide tag/therapeutic agent molecules are covalently linked using a chemical cross-linking agent. Many different cross-linking agents can be used. In certain embodiments the cross-linking agent is about 400-1000 daltons or about 3-12 angstroms in length. The cross-linkers useful in the present invention must be at least bivalent so that they can covalently join two molecules, the homeodomain peptide tag to the therapeutic agent molecule. In certain embodiments, the cross-linker can be tris-succinimidyl aminotriacetate (TSAT); bis(sulfosuccinimidyl)suberate (BS3); disuccinimidyl suberate (DSS); bis (2-[sulfosuccinimidyooxycarbonloxy]ethylsulfone) (BOSCOES); bis(2-[succinimidyooxycarbonloxy]ethylsulfone) (Sulfo-BOSCOES); ethylene glycol bis-(succinimidylsuccinate) (EGS); ethylene glycol bis-(sulfosuccinimidylsuccinate) (Sulfo-EBS); or Dimethyl 3,3′-dithiobis-propionimidate (DTBP). In certain embodiments, the cross-linker is bivalent such as BS3, Sulfo-Boscoes, EGS, Sulfo-EBS, or DTBP.

Methods for attaching cross-linkers are well known in the art (c.f. Hermanson, 1995 Bioconjugate Techniques, Academic Press, Inc. New York, pp. 728; Wong, 1991 Chemistry of Protein Conjugation and Cross-linking. CRC Press, pp. 340; Brinkley, 1992 A brief survey of methods for preparing protein conjugates with dyes, haptens and cross-linking reagents Bioconjugate Chem. 3:2-13).

Examples of suitable linkers include formaldehyde, gluteraldehyde, MBS (m-Maleimidobenzoyl-N-hydroxysuccinimide ester) and/or Sulfo-MBS (the water soluble analog of MBS), etc. Examples of couplers/linkers are described in detail on the world-wide-web at solulink.com/white_papers/peptide and at piercenet.com.

Expression Vectors

According to one aspect, a cell expression system for expressing a homeodomain peptide tag linked to a therapeutic agent in a mammalian recipient is provided. The expression system (also referred to herein as a “genetically modified cell”) comprises a cell and an expression vector for expressing the homeodomain peptide tag linked to a therapeutic agent (i.e., a fusion protein that includes the antibody/protein and the peptide tag).

Expression vectors of the instant invention include, but are not limited to, viruses, plasmids, and other vehicles for delivering heterologous genetic material to cells. Accordingly, the term “expression vector” as used herein refers to a vehicle for delivering heterologous genetic material to a cell. In particular, the expression vector is a recombinant adenoviral, adeno-associated virus (AAV), or lentivirus or retrovirus vector.

The expression vector further includes a promoter for controlling transcription of the heterologous gene. The promoter can be any desired promoter, selected by known considerations, such as the level of expression of a nucleic acid functionally linked to the promoter and the cell type in which the vector is to be used. Promoters can be an exogenous or an endogenous promoter. Promoters can include, for example, known strong promoters such as SV40 or the inducible metallothionein promoter, or an AAV promoter, such as an AAV p5 promoter. Additional examples of promoters include promoters derived from actin genes, immunoglobulin genes, cytomegalovirus (CMV), adenovirus, bovine papilloma virus, adenoviral promoters, such as the adenoviral major late promoter, an inducible heat shock promoter, respiratory syncytial virus, Rous sarcomas virus (RSV), etc. Specifically, the promoter can be AAV2 p5 promoter or AAV4 p5 promoter. Furthermore, smaller fragments of p5 promoter that retain promoter activity can readily be determined by standard procedures including, for example, constructing a series of deletions in the p5 promoter, linking the deletion to a reporter gene, and determining whether the reporter gene is expressed, i.e., transcribed and/or translated. In certain embodiments, the expression vector for expressing the heterologous gene includes an inducible promoter for controlling transcription of the heterologous gene product. Accordingly, delivery of the therapeutic agent in situ is controlled by exposing the cell in situ to conditions, which induce transcription of the heterologous gene.

The expression system is suitable for generating quantities of fusion protein, which can be isolated and/or purified and formed into a therapeutic composition that in certain embodiments is administered to a mammalian recipient.

The expression system is suitable for administration to the mammalian recipient. The expression system may comprise a plurality of non-immortalized genetically modified cells, each cell containing at least one recombinant gene encoding at least one therapeutic agent.

The cell expression system can be formed in vivo. According to yet another aspect, a method for treating a mammalian recipient in vivo is provided. The method includes introducing an expression vector for expressing a heterologous gene product into a cell of the patient in situ, such as via intravenous administration. To form the expression system in vivo, an expression vector for expressing the therapeutic agent is introduced in vivo into the mammalian recipient i.v., where the vector migrates via the vasculature to the brain.

According to yet another aspect of the invention, a method for treating a mammalian recipient in vivo is provided. The method includes introducing an expression vector for expressing a heterologous gene product into a cell of the patient in situ. To form the expression system in vivo, an expression vector for expressing the therapeutic agent is introduced in vivo into target location of the mammalian recipient by, for example, intraperitoneal injection or injection directly into the brain.

The vector further comprises an exogenous (heterologous) nucleic acid functionally linked to the promoter. By “heterologous nucleic acid” is meant that any heterologous or exogenous nucleic acid can be inserted into the vector for transfer into a cell, tissue or organism. The nucleic acid can encode a polypeptide or protein or an antisense RNA, for example. By “functionally linked” is meant such that the promoter can promote expression of the heterologous nucleic acid, as is known in the art, such as appropriate orientation of the promoter relative to the heterologous nucleic acid. Furthermore, the heterologous nucleic acid preferably has all appropriate sequences for expression of the nucleic acid, as known in the art, to functionally encode, i.e., allow the nucleic acid to be expressed. The nucleic acid can include, for example, expression control sequences, such as an enhancer, and necessary information processing sites, such as ribosome binding sites, RNA splice sites, polyadenylation sites, and transcriptional terminator sequences.

The present disclosure also provides a mammalian cell containing a vector described herein. The cell may be human, and may be from brain. The cell type may be a stem or progenitor cell population.

Antigens and Antibodies

“Antigen” refers to a molecule capable of being bound by an antibody. An antigen is additionally capable of being recognized by the immune system and/or being capable of inducing a humoral immune response and/or cellular immune response leading to the activation of B- and/or T-lymphocytes. An antigen can have one or more epitopes (B- and/or T-cell epitopes). Antigens as used herein may also be mixtures of several individual antigens. “Antigenic determinant” refers to that portion of an antigen that is specifically recognized by either B- or T-lymphocytes. B-lymphocytes responding to antigenic determinants produce antibodies, whereas T-lymphocytes respond to antigenic determinants by proliferation and establishment of effector functions critical for the mediation of cellular and/or humoral immunity.

As used herein, the term “antibody” refers to molecules capable of binding an epitope or antigenic determinant. This term includes whole antibodies and antigen-binding fragments thereof, including single-chain antibodies. In certain embodiments, the antibodies are human antigen binding antibody fragments and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. The antibodies can be from any animal origin including birds (e.g. chicken) and mammals (e.g., human, murine, rabbit, goat, guinea pig, camel, horse and the like). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described, for example, in U.S. Pat. No. 5,939,598.

As used herein, the term “monoclonal antibody” refers to an antibody obtained from a group of substantially homogeneous antibodies, that is, an antibody group wherein the antibodies constituting the group are homogeneous except for naturally occurring mutants that exist in a small amount. Monoclonal antibodies are highly specific and interact with a single antigenic site. Furthermore, each monoclonal antibody targets a single antigenic determinant (epitope) on an antigen, as compared to common polyclonal antibody preparations that typically contain various antibodies against diverse antigenic determinants. In addition to their specificity, monoclonal antibodies are advantageous in that they are produced from hybridoma cultures not contaminated with other immunoglobulins.

The adjective “monoclonal” indicates a characteristic of antibodies obtained from a substantially homogeneous group of antibodies, and does not specify antibodies produced by a particular method. For example, a monoclonal antibody to be used in the present invention can be produced by, for example, hybridoma methods (Kohler and Milstein, Nature 256:495, 1975) or recombination methods (U.S. Pat. No. 4,816,567). The monoclonal antibodies used in the present invention can be also isolated from a phage antibody library (Clackson et al., Nature 352:624-628, 1991; Marks et al., J Mol. Biol. 222:581-597, 1991). The monoclonal antibodies of the present invention particularly comprise “chimeric” antibodies (immunoglobulins), wherein a part of a heavy (H) chain and/or light (L) chain is derived from a specific species or a specific antibody class or subclass, and the remaining portion of the chain is derived from another species, or another antibody class or subclass. Furthermore, mutant antibodies and antibody fragments thereof are also comprised in the present invention (U.S. Pat. No. 4,816,567; Morrison et al., Proc. Natl. Acad. Sci. USA 81:6851-6855, 1984).

As used herein, the term “mutant antibody” refers to an antibody comprising a variant amino acid sequence in which one or more amino acid residues have been altered. For example, the variable region of an antibody can be modified to improve its biological properties, such as antigen binding. Such modifications can be achieved by site-directed mutagenesis (see Kunkel, Proc. Natl. Acad. Sci. USA 82: 488 (1985)), PCR-based mutagenesis, cassette mutagenesis, and the like. Such mutants comprise an amino acid sequence which is at least 70% identical to the amino acid sequence of a heavy or light chain variable region of the antibody, more preferably at least 75%, even more preferably at least 80%, still more preferably at least 85%, yet more preferably at least 90%, and most preferably at least 95% identical. As used herein, the term “sequence identity” is defined as the percentage of residues identical to those in the antibody's original amino acid sequence, determined after the sequences are aligned and gaps are appropriately introduced to maximize the sequence identity as necessary.

Methods of Treatment

The present disclosure provides a method of treating a disease such as a genetic disease or cancer in a mammal by administering a polynucleotide, polypeptide, expression vector, or cell described herein. The genetic disease or cancer may be a lysosomal storage disease (LSD) such as infantile or late infantile ceroid lipofuscinoses, Gaucher, Juvenile Batten, Fabry, MLD, Sanfilippo A, Late Infantile Batten, Hunter, Krabbe, Morquio, Pompe, Niemann-Pick C, Tay-Sachs, Hurler (MPS-I H), Sanfilippo B, Maroteaux-Lamy, Niemann-Pick A, Cystinosis, Hurler-Scheie (MPS-I H/S), Sly Syndrome (MPS VII), Scheie (MPS-I S), Infantile Batten, GM1 Gangliosidosis, Mucolipidosis type II/III, or Sandhoff disease.

The genetic disease may be a neurodegenerative disease, such as Huntington's disease, ALS, hereditary spastic hemiplegia, primary lateral sclerosis, spinal muscular atrophy, Kennedy's disease, Alzheimer's disease, a polyglutamine repeat disease, or focal exposure such as Parkinson's disease.

Certain aspects of the disclosure relate to polynucleotides, polypeptides, vectors, and genetically engineered cells (modified in vivo), and the use of them. In particular, the disclosure relates to a method for gene or protein therapy that is capable of both systemic delivery of a therapeutically effective dose of the therapeutic agent.

The mammalian recipient may have a condition that is amenable to gene replacement therapy. As used herein, “gene replacement therapy” refers to administration to the recipient of exogenous genetic material encoding a therapeutic agent and subsequent expression of the administered genetic material in situ. Thus, the phrase “condition amenable to gene replacement therapy” embraces conditions such as genetic diseases (i.e., a disease condition that is attributable to one or more gene defects), acquired pathologies (i.e., a pathological condition which is not attributable to an inborn defect), cancers and prophylactic processes (i.e., prevention of a disease or of an undesired medical condition). Accordingly, as used herein, the term “therapeutic agent” refers to any agent or material, which has a beneficial effect on the mammalian recipient. Thus, “therapeutic agent” embraces both therapeutic and prophylactic molecules having nucleic acid or protein components.

According to one embodiment, the mammalian recipient has a genetic disease and the exogenous genetic material comprises a heterologous gene encoding a therapeutic agent for treating the disease. In yet another embodiment, the mammalian recipient has an acquired pathology and the exogenous genetic material comprises a heterologous gene encoding a therapeutic agent for treating the pathology. According to another embodiment, the patient has a cancer and the exogenous genetic material comprises a heterologous gene encoding an anti-neoplastic agent. In yet another embodiment the patient has an undesired medical condition and the exogenous genetic material comprises a heterologous gene encoding a therapeutic agent for treating the condition.

As used herein, the term “lysosomal enzyme,” a “secreted protein,” a “nuclear protein,” or a “cytoplasmic protein” include variants or biologically active or inactive fragments of these polypeptides. A “variant” of one of the polypeptides is a polypeptide that is not completely identical to a native protein. Such variant protein can be obtained by altering the amino acid sequence by insertion, deletion or substitution of one or more amino acid. The amino acid sequence of the protein is modified, for example by substitution, to create a polypeptide having substantially the same or improved qualities as compared to the native polypeptide. The substitution may be a conserved substitution. A “conserved substitution” is a substitution of an amino acid with another amino acid having a similar side chain. A conserved substitution would be a substitution with an amino acid that makes the smallest change possible in the charge of the amino acid or size of the side chain of the amino acid (alternatively, in the size, charge or kind of chemical group within the side chain) such that the overall peptide retains its spacial conformation but has altered biological activity. For example, common conserved changes might be Asp to Glu, Asn or Gln; His to Lys, Arg or Phe; Asn to Gln, Asp or Glu and Ser to Cys, Thr or Gly. Alanine is commonly used to substitute for other amino acids. The 20 essential amino acids can be grouped as follows: alanine, valine, leucine, isoleucine, proline, phenylalanine, tryptophan and methionine having nonpolar side chains; glycine, serine, threonine, cystine, tyrosine, asparagine and glutamine having uncharged polar side chains; aspartate and glutamate having acidic side chains; and lysine, arginine, and histidine having basic side chains.

The amino acid changes are achieved by changing the codons of the corresponding nucleic acid sequence. It is known that such polypeptides can be obtained based on substituting certain amino acids for other amino acids in the polypeptide structure in order to modify or improve biological activity. For example, through substitution of alternative amino acids, small conformational changes may be conferred upon a polypeptide that results in increased activity. Alternatively, amino acid substitutions in certain polypeptides may be used to provide residues, which may then be linked to other molecules to provide peptide-molecule conjugates which, retain sufficient properties of the starting polypeptide to be useful for other purposes.

One can use the hydropathic index of amino acids in conferring interactive biological function on a polypeptide, wherein it is found that certain amino acids may be substituted for other amino acids having similar hydropathic indices and still retain a similar biological activity. Alternatively, substitution of like amino acids may be made on the basis of hydrophilicity, particularly where the biological function desired in the polypeptide to be generated in intended for use in immunological embodiments. The greatest local average hydrophilicity of a “protein”, as governed by the hydrophilicity of its adjacent amino acids, correlates with its immunogenicity. Accordingly, it is noted that substitutions can be made based on the hydrophilicity assigned to each amino acid.

In using either the hydrophilicity index or hydropathic index, which assigns values to each amino acid, it is preferred to conduct substitutions of amino acids where these values are ±2, with ±1 being particularly preferred, and those with in ±0.5 being the most preferred substitutions.

The variant protein has at least 50%, at least about 80%, or even at least about 90% but less than 100%, contiguous amino acid sequence homology or identity to the amino acid sequence of a corresponding native protein.

The amino acid sequence of the variant polypeptide corresponds essentially to the native polypeptide's amino acid sequence. As used herein “correspond essentially to” refers to a polypeptide sequence that will elicit a biological response substantially the same as the response generated by the native protein. Such a response may be at least 60% of the level generated by the native protein, and may even be at least 80% of the level generated by native protein.

A variant may include amino acid residues not present in the corresponding native protein or deletions relative to the corresponding native protein. A variant may also be a truncated “fragment” as compared to the corresponding native protein, i.e., only a portion of a full-length protein. Protein variants also include peptides having at least one D-amino acid.

The variant protein may be expressed from an isolated DNA sequence encoding the variant protein. “Recombinant” is defined as a peptide or nucleic acid produced by the processes of genetic engineering. It should be noted that it is well-known in the art that, due to the redundancy in the genetic code, individual nucleotides can be readily exchanged in a codon and still result in an identical amino acid sequence.

The terms “protein,” “peptide” and “polypeptide” are used interchangeably herein.

The present disclosure provides methods of treating a disease in a mammal by administering an expression vector to a cell or patient. For the gene therapy methods, a person having ordinary skill in the art of molecular biology and gene therapy would be able to determine, without undue experimentation, the appropriate dosages and routes of administration of the expression vector used in the novel methods of the present disclosure.

According to one embodiment, the cells are transformed or otherwise genetically modified in vivo. The cells from the mammalian recipient are transformed (i.e., transduced or transfected) in vivo with a vector containing exogenous genetic material for expressing a heterologous (e.g., recombinant) gene encoding a therapeutic agent and the therapeutic agent is delivered in situ.

As used herein, “exogenous genetic material” refers to a nucleic acid or an oligonucleotide, either natural or synthetic, that is not naturally found in the cells; or if it is naturally found in the cells, it is not transcribed or expressed at biologically significant levels by the cells. Thus, “exogenous genetic material” includes, for example, a non-naturally occurring nucleic acid that can be transcribed into anti-sense RNA, as well as a “heterologous gene” (i.e., a gene encoding a protein which is not expressed or is expressed at biologically insignificant levels in a naturally-occurring cell of the same type).

In the certain embodiments, the mammalian recipient has a condition that is amenable to gene replacement therapy. As used herein, “gene replacement therapy” refers to administration to the recipient of exogenous genetic material encoding a therapeutic agent and subsequent expression of the administered genetic material in situ. Thus, the phrase “condition amenable to gene replacement therapy” embraces conditions such as genetic diseases (i.e., a disease condition that is attributable to one or more gene defects), acquired pathologies (i.e., a pathological condition which is not attributable to an inborn defect), cancers and prophylactic processes (i.e., prevention of a disease or of an undesired medical condition). Accordingly, as used herein, the term “therapeutic agent” refers to any agent or material, which has a beneficial effect on the mammalian recipient. Thus, “therapeutic agent” embraces both therapeutic and prophylactic molecules having nucleic acid (e.g., antisense RNA) and/or protein components.

Alternatively, the condition amenable to gene replacement therapy is a prophylactic process, i.e., a process for preventing disease or an undesired medical condition. Thus, the instant disclosure embraces a cell expression system for delivering a therapeutic agent that has a prophylactic function (i.e., a prophylactic agent) to the mammalian recipient.

In summary, the term “therapeutic agent” includes, but is not limited to, agents associated with the conditions listed above, as well as their functional equivalents. As used herein, the term “functional equivalent” refers to a molecule (e.g., a peptide or protein) that has the same or an improved beneficial effect on the mammalian recipient as the therapeutic agent of which is it deemed a functional equivalent.

The above-disclosed therapeutic agents and conditions amenable to gene replacement therapy are merely illustrative and are not intended to limit the scope of the instant disclosure. The selection of a suitable therapeutic agent for treating a known condition is deemed to be within the scope of one of ordinary skill of the art without undue experimentation.

Suitable methods for the delivery and introduction of transduced cells into a subject have been described. For example, cells can be transduced in vitro by combining recombinant AAV virions with CNS cells e.g., in appropriate media, and screening for those cells harboring the DNA of interest can be screened using conventional techniques such as Southern blots and/or PCR, or by using selectable markers. Transduced cells can then be formulated into pharmaceutical compositions, described more fully below, and the composition introduced into the subject by various techniques, such as by grafting, intramuscular, intravenous, subcutaneous and intraperitoneal injection.

In one embodiment, pharmaceutical compositions will comprise sufficient genetic material to produce a therapeutically effective amount of the nucleic acid of interest, i.e., an amount sufficient to reduce or ameliorate symptoms of the disease state in question or an amount sufficient to confer the desired benefit. The pharmaceutical compositions will also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., N.J. 1991).

As is apparent to those skilled in the art in view of the teachings of this specification, an effective amount of viral vector which must be added can be empirically determined. Administration can be effected in one dose, continuously or intermittently throughout the course of treatment. Methods of determining the most effective means and dosages of administration are well known to those of skill in the art and will vary with the viral vector, the composition of the therapy, the target cells, and the subject being treated. Single and multiple administrations can be carried out with the dose level and pattern being selected by the treating physician.

It should be understood that more than one transgene could be expressed by the delivered viral vector. Alternatively, separate vectors, each expressing one or more different transgenes, can also be delivered to the CNS as described herein. Furthermore, it is also intended that the viral vectors delivered by the methods of the present disclosure be combined with other suitable compositions and therapies.

Formulations and Methods of Administration

The compositions of the invention may be formulated as pharmaceutical compositions (e.g., comprising fusion proteins or expression vectors) and administered to a mammalian host, such as a human patient, in a variety of forms adapted to the chosen route of administration, i.e., orally, intranasally, intradermally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course; be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with several of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions. For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions that can be used to deliver the compounds of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of the present invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Methods for Introducing Genetic Material into Cells

The present method provides a method of delivering a nucleic acid to a cell comprising administering to the cell a vector comprising the nucleic acid encoding the therapeutic agent, thereby delivering the nucleic acid to the cell. The cells can include any desired cell in humans as well as other large (non-rodent) mammals, such as primates, horse, sheep, goat, pig, and dog. In certain embodiments, the present invention provides a method of delivering a nucleic acid to a brain cell, comprising administering to the brain cell a vector comprising the nucleic acid encoding a therapeutic agent, thereby delivering the nucleic acid to the brain cell.

The present invention also includes a method of delivering a nucleic acid to a subject comprising administering to a cell from the subject vector comprising the nucleic acid encoding the therapeutic agent, and returning the cell to the subject, thereby delivering the nucleic acid to the subject. For such an ex vivo administration, cells are isolated from a subject by standard means according to the cell type and placed in appropriate culture medium, again according to cell type. Viral particles are then contacted with the cells as described above, and the virus is allowed to transfect the cells. Cells can then be transplanted back into the subject's body, again by means standard for the cell type and tissue. If desired, prior to transplantation, the cells can be studied for degree of transfection by the virus, by known detection means and as described herein.

The exogenous genetic material (e.g., a cDNA encoding one or more therapeutic proteins) is introduced into the cell ex vivo or in vivo by genetic transfer methods, such as transfection or transduction, to provide a genetically modified cell. Various expression vectors e., vehicles for facilitating delivery of exogenous genetic material into a target cell) are known to one of ordinary skill in the art.

As used herein, “transfection of cells” refers to the acquisition by a cell of new genetic material by incorporation of added DNA. Thus, transfection refers to the insertion of nucleic acid into a cell using physical or chemical methods. Several transfection techniques are known to those of ordinary skill in the art including: calcium phosphate DNA co-precipitation; DEAE-dextran; electroporation; cationic liposome-mediated transfection; and tungsten particle-faciliated microparticle bombardment. Strontium phosphate DNA co-precipitation is another possible transfection method.

In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous genetic material contained within the retrovirus is incorporated into the genome of the transduced cell. A cell that has been transduced with a chimeric DNA virus (e.g., an adenovirus carrying a cDNA encoding a therapeutic agent), will not have the exogenous genetic material incorporated into its genome but will be capable of expressing the exogenous genetic material that is retained extrachromosomally within the cell.

Typically, the exogenous genetic material includes the heterologous gene (usually in the form of a cDNA comprising the exons coding for the therapeutic protein) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any non-translated DNA sequence which works contiguous with the coding sequence (in cis) to change the basal transcription level dictated by the promoter. The exogenous genetic material may introduced into the cell genome immediately downstream from the promoter so that the promoter and coding sequence are operatively linked so as to permit transcription of the coding sequence. A retroviral expression vector may include an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eucaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. If the gene encoding the therapeutic agent is under the control of an inducible promoter, delivery of the therapeutic agent in situ is triggered by exposing the genetically modified cell in situ to conditions for permitting transcription of the therapeutic agent, e.g., by intraperitoneal injection of specific inducers of the inducible promoters which control transcription of the agent. For example, in situ expression by genetically modified cells of a therapeutic agent encoded by a gene under the control of the metallothionein promoter, is enhanced by contacting the genetically modified cells with a solution containing the appropriate (i.e., inducing) metal ions in situ.

Accordingly, the amount of therapeutic agent that is delivered in situ is regulated by controlling such factors as: (1) the nature of the promoter used to direct transcription of the inserted gene, (i.e., whether the promoter is constitutive or inducible, strong or weak); (2) the number of copies of the exogenous gene that are inserted into the cell; (3) the number of transduced/transfected cells that are administered (e.g., implanted) to the patient; (4) the size of the implant (e.g., graft or encapsulated expression system); (5) the number of implants; (6) the length of time the transduced/transfected cells or implants are left in place; and (7) the production rate of the therapeutic agent by the genetically modified cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector may include a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene and/or signal sequence (described below) is deemed to be within the scope of one of ordinary skill in the art without undue experimentation.

The therapeutic agent can be targeted for delivery to an extracellular, intracellular or membrane location. If it is desirable for the gene product to be secreted from the cells, the expression vector is designed to include an appropriate secretion “signal” sequence for secreting the therapeutic gene product from the cell to the extracellular milieu. If it is desirable for the gene product to be retained within the cell, this secretion signal sequence is omitted. In a similar manner, the expression vector can be constructed to include “retention” signal sequences for anchoring the therapeutic agent within the cell plasma membrane. For example, all membrane proteins have hydrophobic transmembrane regions, which stop translocation of the protein in the membrane and do not allow the protein to be secreted. The construction of an expression vector including signal sequences for targeting a gene product to a particular location is deemed to be within the scope of one of ordinary skill in the art without the need for undue experimentation.

Certain embodiments of the present disclosure provide a cell comprising a viral vector as described herein. In certain embodiments, the cell is a mammalian cell of a non-rodent mammal. In certain embodiments, the cell is a primate cell. In certain embodiments, the cell is a human cell. In certain embodiments, the cell is a non-human cell. In certain embodiments, the cell is in vitro. In certain embodiments, the cell is in vivo. In certain embodiments, the cell is a brain cell.

Certain embodiments of the present disclosure provide a method of treating a disease in a mammal comprising administering a viral vector or the cell as described herein to the mammal. In certain embodiments, the mammal is human.

In certain embodiments, the disease is a lysosomal storage disease (LSD). In certain embodiments, the LSD is infantile or late infantile ceroid lipofuscinoses, Gaucher, Juvenile Batten, Fabry, MLD, Sanfilippo A, Late Infantile Batten, Hunter, Krabbe, Morquio, Pompe, Niemann-Pick C, Tay-Sachs, Hurler (MPS-I H), Sanfilippo B, Maroteaux-Lamy, Niemann-Pick A, Cystinosis, Hurler-Scheie (MPS-I H/S), Sly Syndrome (MPS VII), Scheie (MPS-I S), Infantile Batten, GM1 Gangliosidosis, Mucolipidosis type or Sandhoff disease.

In certain embodiments, the disease is a neurodegenerative disease. In certain embodiments, the neurodegenerative disease is Huntington's disease, ALS, hereditary spastic hemiplegia, primary lateral sclerosis, spinal muscular atrophy, Kennedy's disease, Alzheimer's disease, a polyglutamine repeat disease, or Parkinson's disease.

Certain embodiments of the present disclosure provide a protein, a viral vector or cell as described herein for use in medical treatments.

Certain embodiments of the present disclosure provide a use of a protein, a viral vector or cell as described herein to prepare a medicament useful for treating a disease in a mammal.

Definitions

“Bound” refers to binding or attachment that may be covalent, e.g., by chemically coupling, or non-covalent, e.g., ionic interactions, hydrophobic interactions, hydrogen bonds. Covalent bonds can be, for example, ester, ether, phosphoester, amide, peptide, imide, carbon-sulfur bonds, carbon-phosphorus bonds, and the like. The term “bound” is broader than and includes terms such as “conjugated” “coupled,” “fused” and “attached.”

The term “polypeptide” as used herein refers to a polymer of amino acids and includes full-length proteins and fragments thereof. Thus, “protein,” polypeptide,” and “peptide” are often used interchangeably herein. Substitutions can be selected by known parameters to be neutral. As will be appreciated by those skilled in the art, the invention also includes those polypeptides having slight variations in amino acid sequences or other properties. Such variations may arise naturally as allelic variations (e.g. due to genetic polymorphism) or may be produced by human intervention (e.g., by mutagenesis of cloned DNA sequences), such as induced point, deletion, insertion and substitution mutants. Minor changes in amino acid sequence are generally preferred, such as conservative amino acid replacements, small internal deletions or insertions, and additions or deletions at the ends of the molecules. These modifications can result in changes in the amino acid sequence, provide silent mutations, modify a restriction site, or provide other specific mutations.

The invention encompasses isolated or substantially purified protein compositions. In the context of the present invention, an “isolated” or “purified” polypeptide is a polypeptide that exists apart from its native environment and is therefore not a product of nature. A polypeptide may exist in a purified form or may exist in a non-native environment such as, for example, a transgenic host cell. For example, an “isolated” or “purified” protein, or biologically active portion thereof, is substantially free of other cellular material, or culture medium when produced by recombinant techniques, or substantially free of chemical precursors or other chemicals when chemically synthesized. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed proteins or partial-length proteins encoded thereby are also encompassed by the present invention. By “fragment” or “portion” is meant a full length or less than full length of the amino acid sequence of, a polypeptide or protein.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

A “variant” of a molecule is a sequence that is substantially similar to the sequence of the native molecule.

“Wild-type” refers to the normal gene, or organism found in nature without any known mutation. “Operably-linked” refers to the association of molecules so that the function of one is affected by the other. Nucleic acid is “operably linked” when it is placed into a functional relationship with another nucleic acid sequence. Generally, “operably linked” means that the DNA sequences being linked are contiguous. However, enhancers do not have to be contiguous. Linking is accomplished by ligation at convenient restriction sites. If such sites do not exist, the synthetic oligonucleotide adaptors or linkers are used in accordance with conventional practice. Additionally, multiple copies of the nucleic acid encoding enzymes may be linked together in the expression vector. Such multiple nucleic acids may be separated by linkers.

The invention will now be illustrated by the following non-limiting Example.

### EXAMPLE 1

Morphology Specific Anti-Synuclein Nanobodies as Therapeutics for PD

Experiment 1: Lentiviral delivery of different nanobodes in an a-syn tg mouse model for long-term therapy. This portion of the project examined the ability of the lentivirus to deliver the nanobody to the CNS and to effectively reduce the burden of a-syn in a tg mouse model of PD. These studies identified the two most promising nanobody constructs for further testing by iv injection. For these experiments, the line 61 a-syn tg model of PD was used, which has a-syn inclusions beginning at 3-4 months of age (Rockenstein, E., et al., Differential neuropathological alterations in transgenic mice expressing alpha-synuclein from the platelet-derived growth factor and Thy-1 promoters. J Neurosci Res, 2002. 68: p. 568-78). This leads to reduced TH immunoreactivity and reduced performance on the rotorod and inverted pole tests.

Experiment 2: Study the ability of nanobodies targeted to a-syn to cross into the CNS and reduce a-syn accumulation in transgenic mice. The purpose of this experiment was to assess the ability of the two most protective nanobody constructs identified in Experiment 1 to reduce the accumulation of a-syn in a mouse model of PD when injected by iv administration. Nanobodies were purified and delivered to mice by iv tail injection. Mice were examined for the extent of delivery of nanobody and for its ability to reduce the levels of a-syn and effects on behavior.

Different constructs for the lentiviruses were constructed. The nanobody constructs included D10, which binds all forms of a-syn and D5 and 10H which recognizes two different oligomeric forms. In addition, we generated a tagged version of D10, D5 and 10H containing a homeodomain secretion/penetration tag. Construction of the tagged versions of the nanobodies took longer than expected due to difficulties with the PCR reactions to add the secretion tag to the different nanobodies, but these issues were resolved.

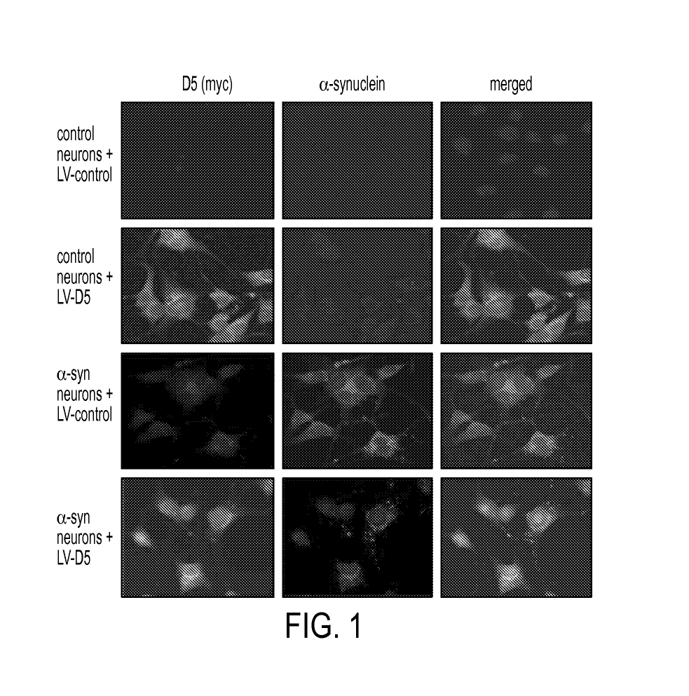

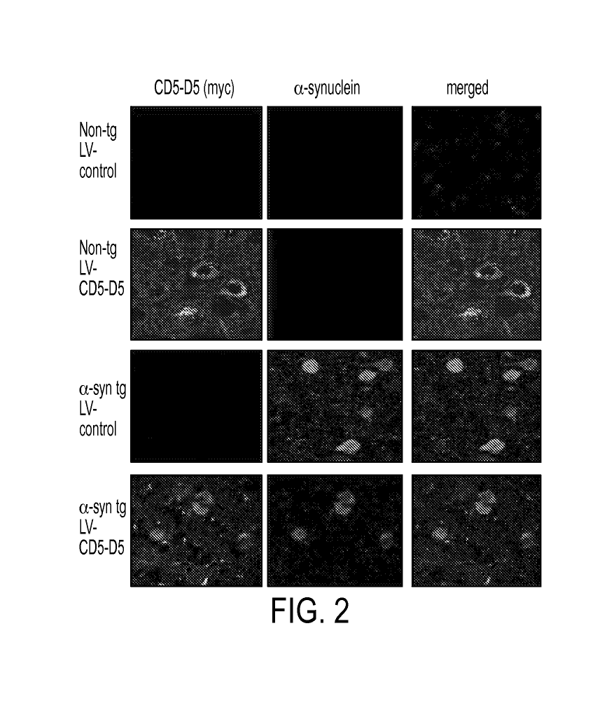

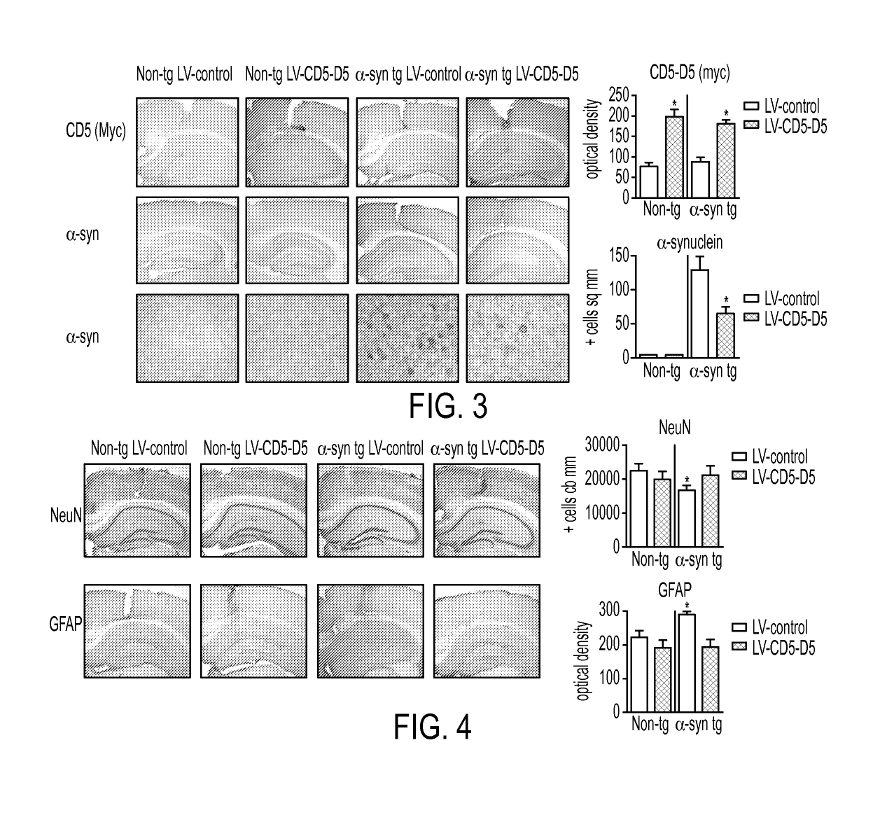

Lentiviral constructs with D5 and D10 were made using two variants of each one, one with a traditional antibody secretion signal (cd5), and one without. Four test groups (D5, D5-cd5, D10, D10-cd5), and a lentiviral control (LV) were established. An average of six mice per group were injected, including a-synuclein tg and non-tg age 8-10 m. Neuropathological analysis after the LV injections were performed. The secreted version of D5 (LV-cd5-D5) was shown to be the most effective at reducing the accumulation of a-syn and neurodegeneration in the 8-10 month tg mice. In studies using the non-secreted version of D5, the D5 nanobody is accumulated intracellularly in transfected neurons, and lowered intracellular a-syn levels compared to the tg mice receiving a control vector (FIG. 1). Similar studies using the secreted version of D5 showed little intracellular accumulation of D5, and significantly less accumulation of intracellular a-syn compared to the tg mice receiving the control vector (FIG. 2). Analysis of brain slices from the wt and tg mice treated with either the secreted version of D5 or with a control virus showed presence of D5 in the brain tissue of mice treated with the D5 vector. Significant a-syn aggregation was observed in the tg mouse brain tissue treated with control vector, but substantially reduced aggregation was observed in the tg mouse treated with cd5-D5 (FIG. 3).

Finally, the presence of two neuronal markers, NeuN and GFAP to assess the presence of neuronal and glial cells was also performed. NeuN staining was unchanged in the wt mice treated with either control or cd5-D5 virus. However NeuN levels were reduced in the tg mouse tissue treated with control virus compared to the wt mice, but returned to wt levels in the tg mice treated with cd5-D5 (FIG. 4). GFAP was used to assess the level of glial cells in the brain tissue. GFAP levels were unchanged in the wt mice treated with the control and cd5-D5 virus treatment, potentially decreasing slightly with the cd5- D5 tissue. However GFAP levels substantially increased in the tg mouse tissue treated with control virus compared to the wt mice. GFAP levels in the tg mouse tissue treated with cd5-D5 were similar to wt levels (FIG. 4). These encouraging results indicate that the D5 nanobody can decrease a-syn aggregation in mouse models of PD, and restore neuronal health.

Since the data obtained showed that the oligomeric D5 nanobody protected better than the pan-specific D10 nanobody in mouse models, further studies focused on the two oligomer specific nanobodies, D5 and 10H. Note that these studies are in agreement with earlier studies in cell models of Parkinson's disease (PD) which also showed that D5 provided better protection from a-syn induced toxicity compared to D10.

Next, experiments were performed to determine which nanobody construct showed the best protection in PD mice utilizing i.p. lentiviral injection. This method of viral injection resulted in transduction of liver and spleen cells. The transduced cells secreted the different nanobody constructs where they could circulate in the blood stream and cross the blood brain barrier (BBB) to enter the brain. The goal of this study was to identify which construct could best cross the BBB and reduce a-syn pathology and improve behavior in the PD mouse model. Three different constructs were tested, D5 and 10H both containing a homeodomain secretion/penetration peptide tag to facilitate transfer across the BBB, and a control empty lentivirus (Bobi). The lentiviral constructs were injected into both wild-type and control mice at three different time points: 3, 6 and 9 months. Mice underwent behavior analysis and after two months of treatment were sacrificed for pathology analysis. The number of surviving mice from each group are shown in Table 1.

Behavioral testing. Mice were subjected to three different behavior tests, an adhesive removal test (ART), a grip test and a hanging wire test.

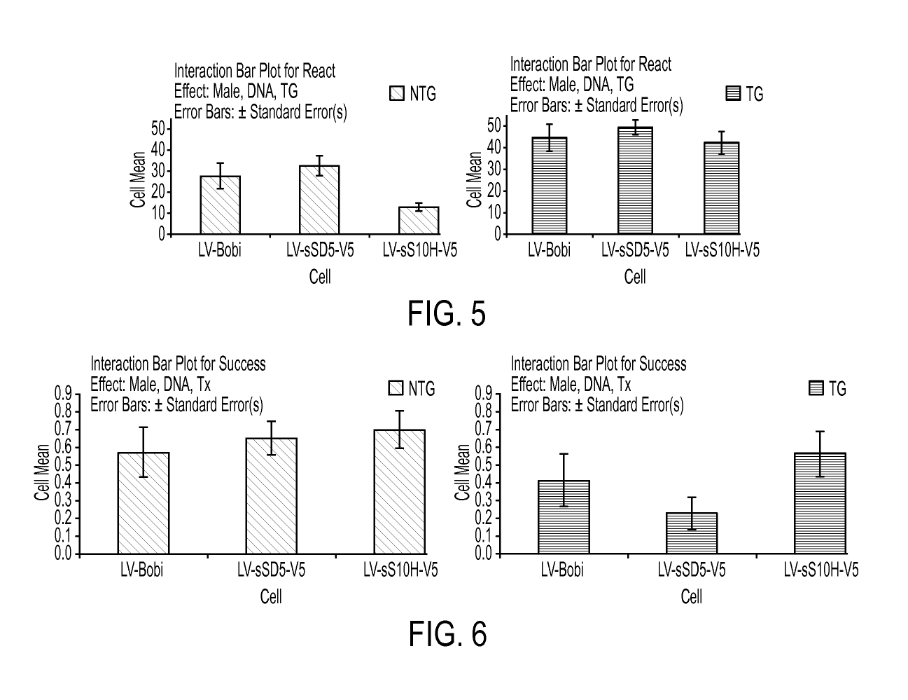

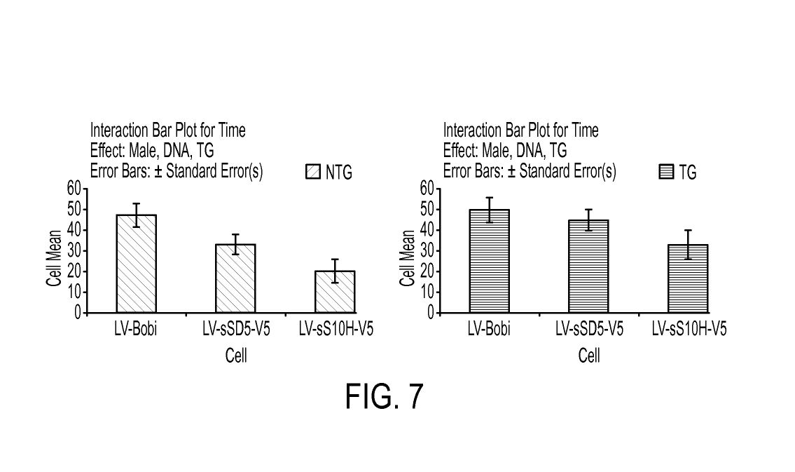

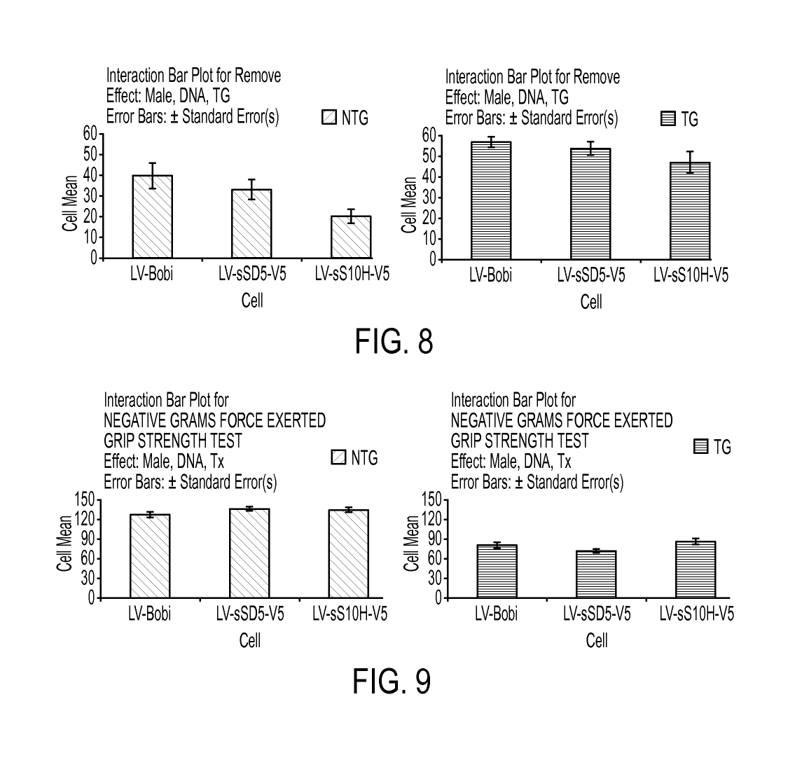

Adhesive Removal Test (ART). Mice were tested for React, success, and Time and Removal. Results from these analyses are shown in FIGS. 5-8. In general, 10H showed improvement in most tests compared to Bobi in both the wt and tg mice. D5 also showed improvement in most tests, but not as much as 10H.

Grip Strength Test. Results of grip strength test are shown in FIG. 9. Mice treated with 10H showed an increased grip strength compared to Bobi in both wt and tg mice. Mice treated with D5 showed an increase in grip strength in the wt mice, but a slight decrease in the tg mice.

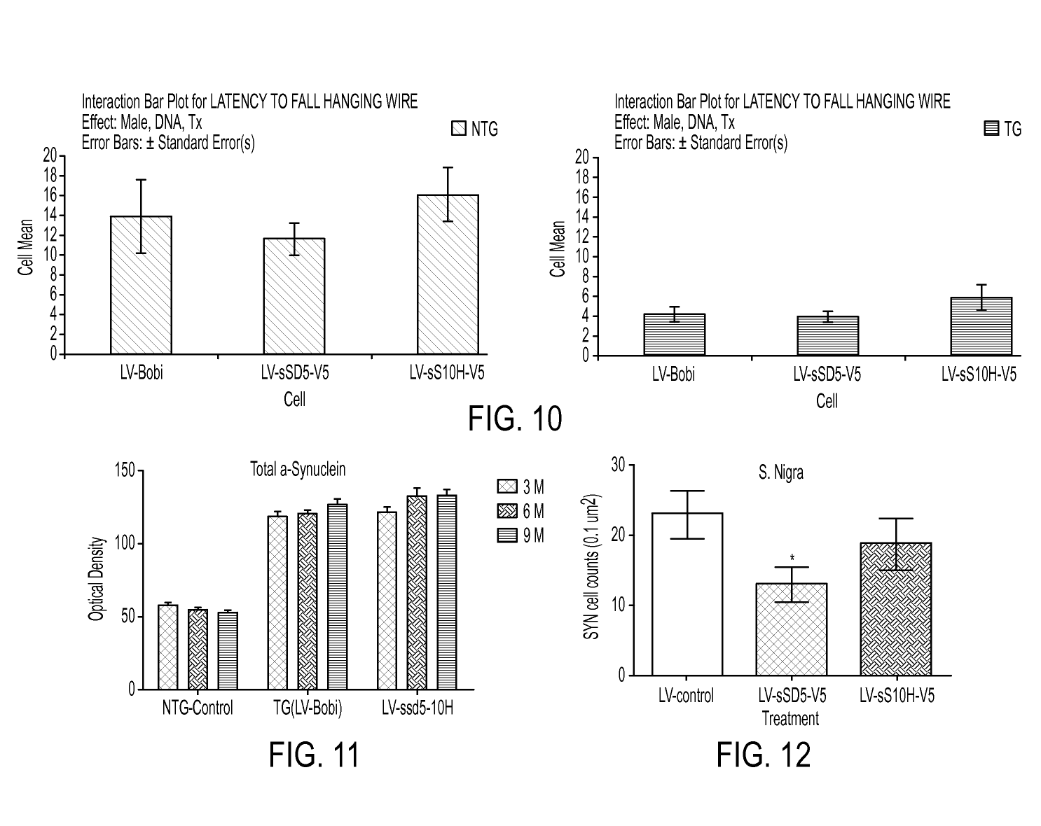

Hanging Wire test. Results of the hanging wire test are shown in FIG. 10. Mice treated with 10H again showed improvement compared to Bobi in both wt and tg mice. Mice treated with D5 showed no change compared to mice treated with Bobi.

Pathology. Brain tissue from all mice was tested for the presence of total a-synuclein levels, various different aggregated forms of a-synuclein including proteinase K resistant, fibrillar and different oligomeric a-syn species.

Total a-syn levels. Total a-syn levels in brain tissue of selected 3, 6, and 9 month mice were determined by immunoreactivity or brain slices with D10, an antibody that recognizes all forms of a-syn. The analysis indicates that the tg mice have greatly substantially higher levels of a-syn compared to wt mice as expected and that while the levels increase slightly from 3 to 9 months in the tg mice, total a-syn levels remain essentially constant in the D5 treated and control treated mice. (See FIG. 11).

Proteinase K resistant a-syn inclusions in the Substantia Nigra. Levels of proteins K resistant a-syn inclusions in the substantia nigra decreased in both the tg D5 and 10H treated mice, although significantly more in the D5 treated mice (FIG. 12).

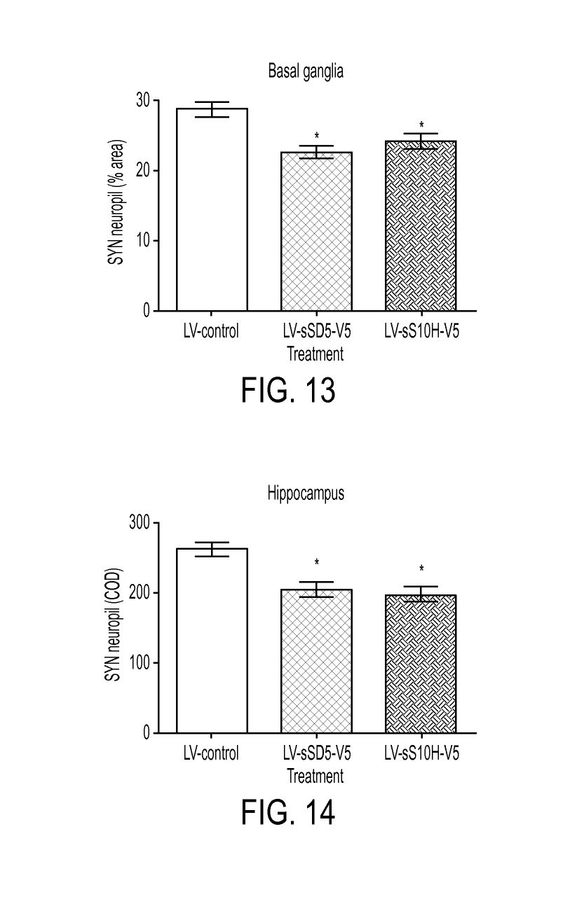

Synuclein axonal dystrophy in basal ganglia. The levels of synuclein axonal dystrophy in the basal ganglia also decreased in the tg D5 and 10H treated mice compared to the control, with D5 again having lower levels than 10H (FIG. 13).

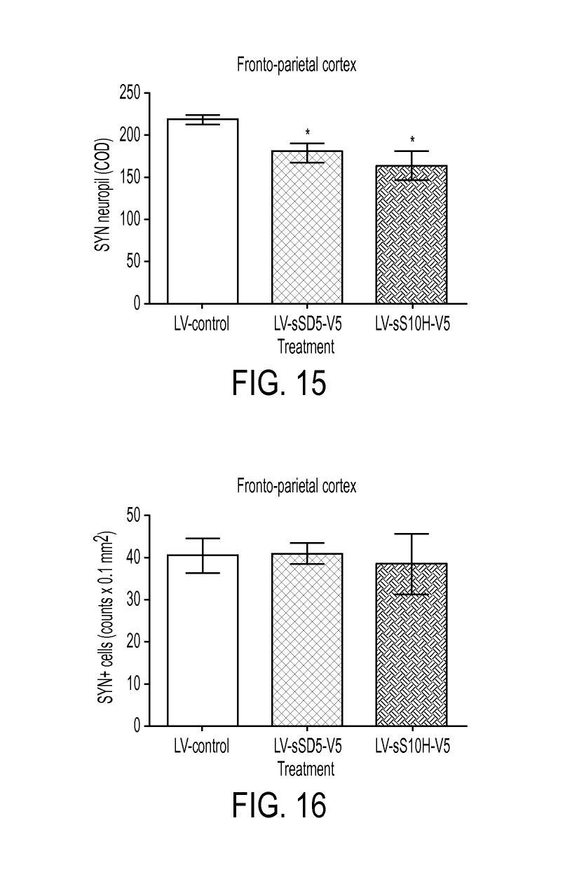

Synuclein containing neuropils. Synuclein containing neuropils in the hippocampus also decreased in the tg D5 and 10H treated mice, although to equal levels compared to the control (FIG. 14). Synuclein containing neuropils in the fronto-parietal cortex also decreased in the treated mice, with a slightly larger decrease obtained in the 10H treated mice (FIG. 15). The total number of synuclein positive cells in the fronto-parietal cortex remained the same in all the tg mice (FIG. 16) indicating that a-syn expression has not changed.

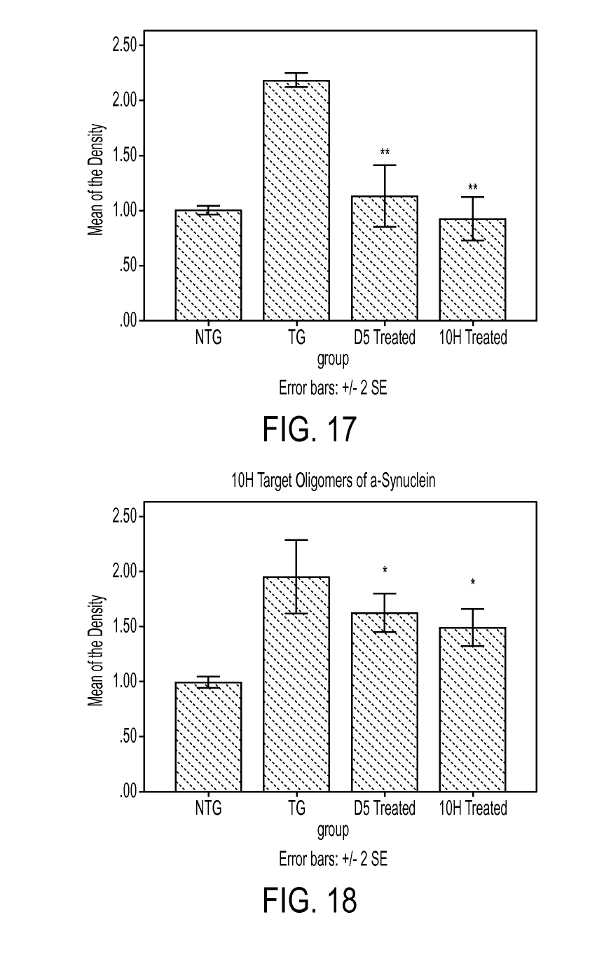

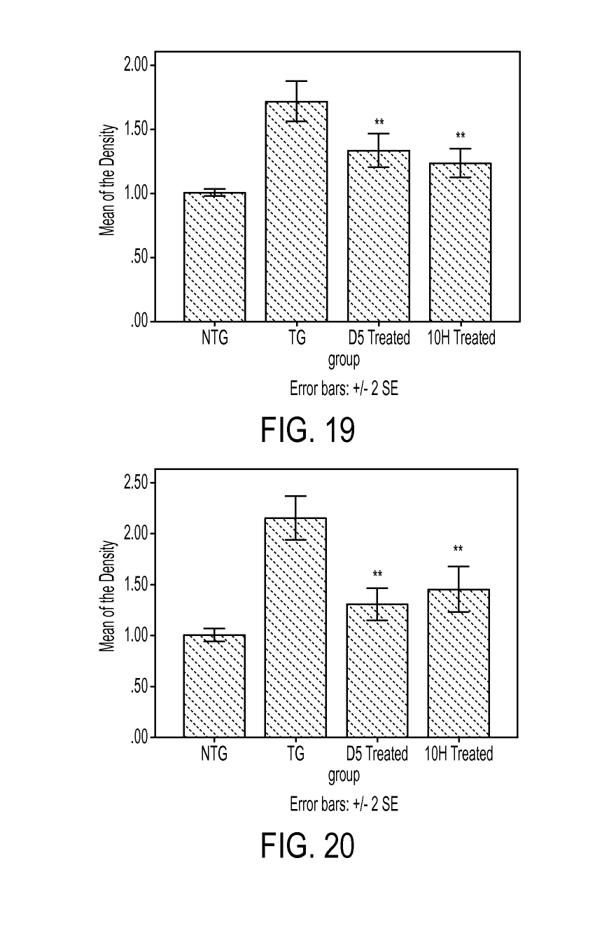

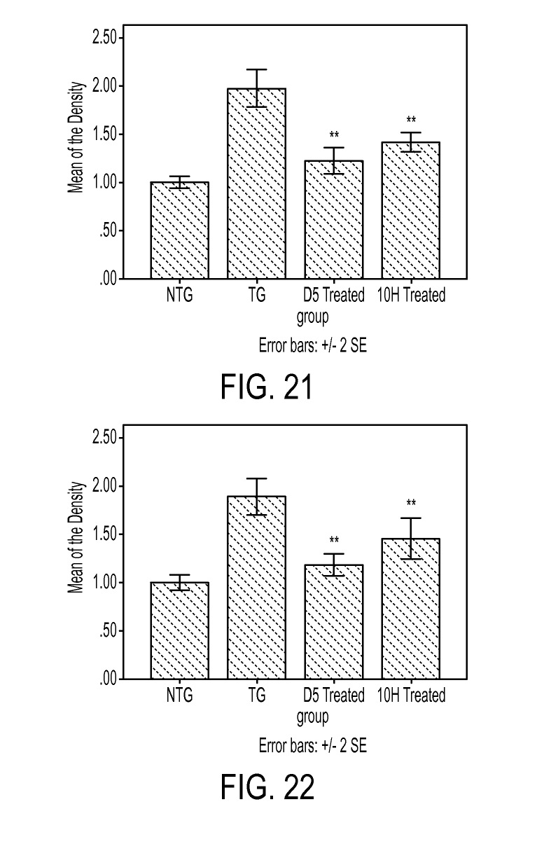

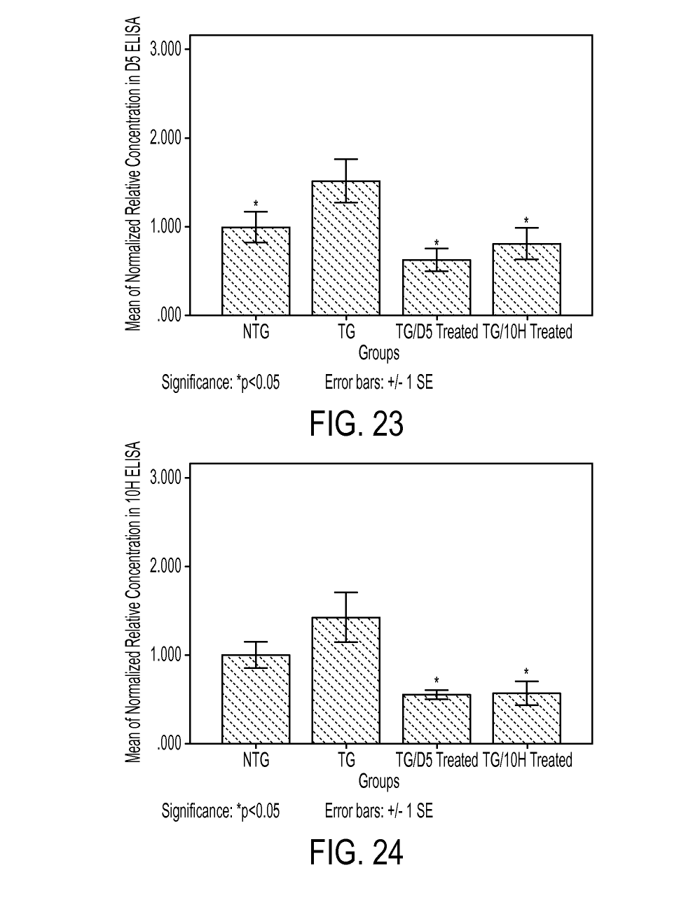

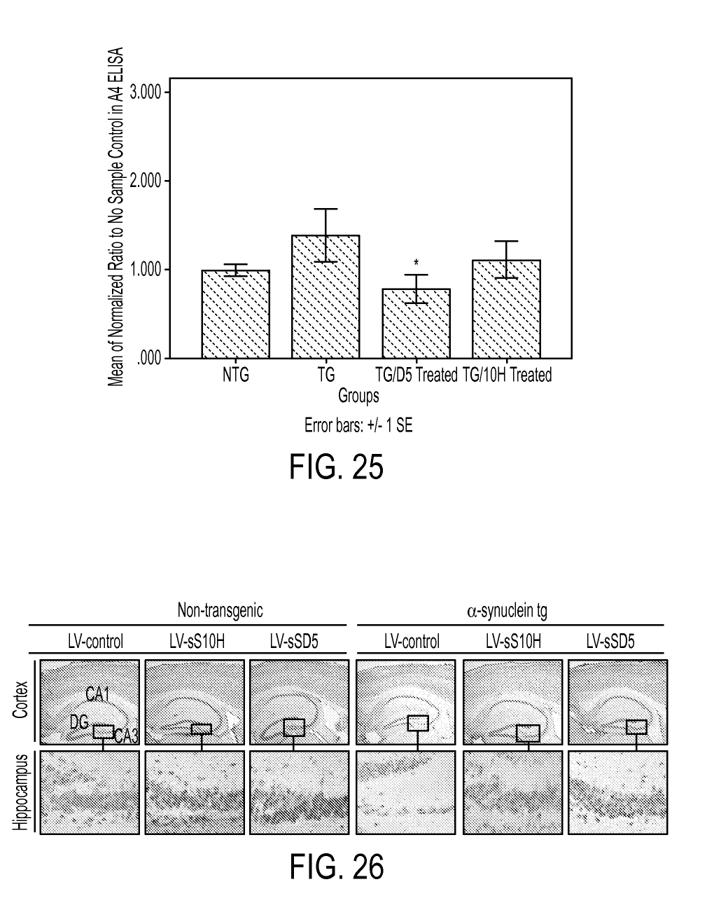

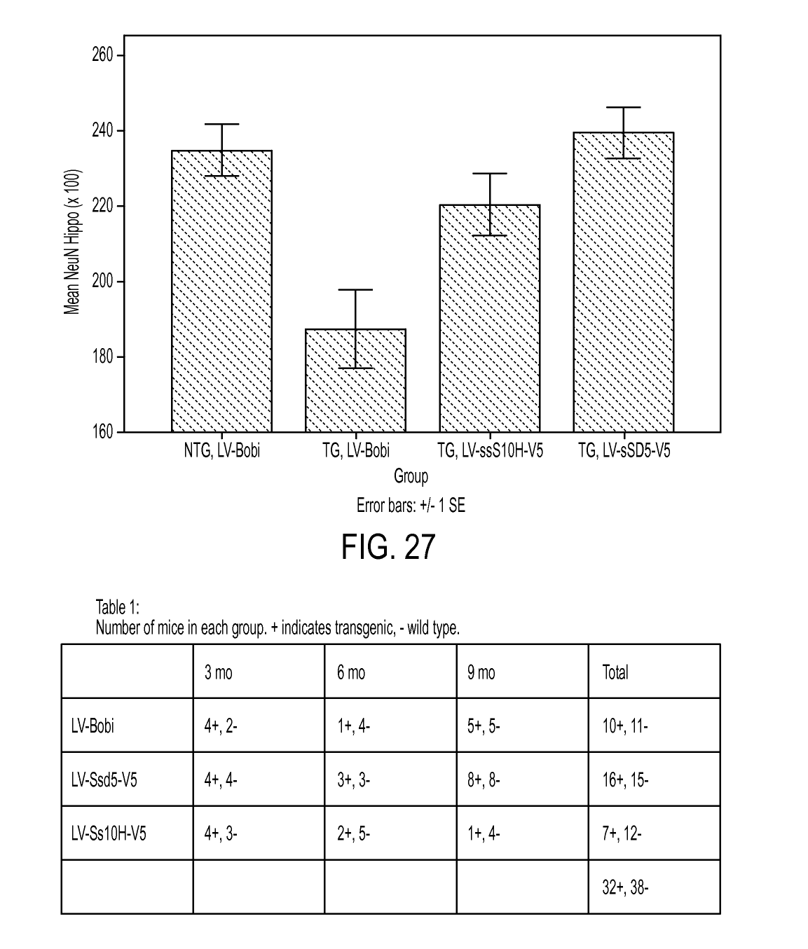

Oligomeric a-syn levels. The presence of both D5 and 10H reactive oligomeric a-syn in the treated and control tg mice were tested by immunohistochemistry. Levels of D5 reactive oligomeric a-syn decreased substantially in both the 10H and D5 treated mice (FIG. 17) as did levels of 10H reactive oligomeric a-syn (FIG. 18). The levels of D5 decreased substantially in both the cortex (FIG. 19) and hippocampus (FIG. 20) of the treated mice as did levels of 10H (FIGS. 21 and 22) We also tested levels of D5 reactive oligomeric a-syn in homogenized mouse brain tissue samples by ELISA. Levels of D5 reactive oligomeric a-syn were substantially reduced in both the 10H and D5 treated mice, with levels in the D5 treated mice decreasing even below those found in wt control mice (FIG. 23). Similar results were obtained when testing for levels of 10H reactive oligomeric a-syn (FIG. 24). We also tested for levels of a toxic oligomeric beta-amyloid species. Again D5 treated mice showed substantially lower levels of oligomeric beta-amyloid compared to the control tg and wt mice, where 10H showed lower levels than the tg control but similar levels to wt mice (FIG. 25).

Degeneration of neuronal cells in the hippocampus and temporal cortex was also tested. The Thy 1 mouse model shows a reduction of cells in the CA3 region of the hippocampus and temporal cortex. Treatment with D5 and 10H showed a substantial increase in number of cells in the tg mice compared to control and also a potentially small increase in cell numbers in the wt treated mice compared to the wt control (FIG. 26). Complete analysis of the samples is still in progress. Again while both D5 and 10H treated mice show improvement in cells in the hippocampus, cell levels in mice treated with D5 are slightly better than in those treated with 10H. These results indicate that treatment with D5 and 10H results in generation of new neurons in critically affected areas of the PD mouse brain.

Conclusion

It was studied whether the nanobodies, D5 and 10H, which selectively recognize different oligomeric a-syn species, can provide protection against a-syn induced pathology in a mouse model of PD. While D5 and 10H recognize different naturally occurring oligomeric a-syn species, it is important to note that they do not interact with monomeric or fibrillar forms, and since the nanobodies only contain the antibody binding domain, will not activate an immune response. Therefore any effects observed are due to specific interactions with the different oligomeric a-syn aggregates. Expression of both anti-oligomeric a-syn antibody fragments in a tg PD mouse model showed significant improvement in neuronal health and pathology. Direct intracranial injection of lentivirus expressing either D5 or 10H showed a decrease in a-syn pathology and restoration of neuronal markers to wild-type levels. When the nanobodies were expressed in the liver by ip injection of lentivirus, expression of D5 and 10H again showed similar protection against a-syn pathology. The nanobodies contained a homeodomain secretion/penetration (sec/pen) tag to facilitate transport across the BBB and to allow the nanobodies to enter and exit cells. When expressed in the blood stream, both nanobodies showed substantial reduction in a-syn pathology, though D5 generally showed a more significant reduction in pathology compared to 10H. The total amount of a-syn produced in the tg animals was not altered in the treated mice, nor were the number of a-syn producing cells. However the amount of PK resistant a-syn aggregates, fibrillar a-syn, and both D5 and 10H reactive oligomeric a-syn aggregates were quite dramatically reduced in the D5 and 10H treated mice. In the case of the D5 treated mice, the levels of a-syn pathology were generally similar or below those found in wt mice. Levels of toxic oligomeric beta-amyloid were also reduced in the treated mice compared to the tg control, where again the D5 treated mice decreased levels of oligomeric beta-amyloid to below that found in the wt control mice. Tg mice treated with D5 and 10H also showed a substantial increase in the number of cells in the hippocampus and temporal gyms, areas that are affected in this mouse model, indicating that treatment with these antibodies helps restore neurons in affected brain regions. While both D5 and 10H showed improvement in number of cells, D5 again showed better improvement. All these positive results in brain pathology in the D5 and 10H treated mice were obtained without affecting levels of monomeric a-syn. The 10H treated mice also showed some improvement in behavioral testing, showing similar improvements in both the tg and wt treated mice. Both D5 and 10H are designed to specifically bind only to toxic oligomeric a-syn species to minimize any potential unwanted side effects and to maintain the function of monomeric a-syn.

The results obtained here indicate that both sec/pen tagged D5 and 10H nanobodies effectively cross the BBB and reduce a-syn pathology in a PD mouse model. Since different oligomeric forms of a-syn occur in different cellular locations and have different toxic mechanisms, D5 and 10H may target and block different toxic pathways in different places, potentially explaining why the two nanobodies both protect against a-syn pathology, but in different ways. However, both D5 and 10H provide excellent protection against a-syn pathology in PD mouse models, with no readily apparent negative side effects in the wt mice, but rather with potentially positive effects in the wt mice as well. Since they target different oligomeric species, a combination of both D5 and 10H represent the most promising therapeutic.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of delivering a protein to the brain of a mammal, comprising administering to the mammal
(a) a therapeutic fusion protein comprising a homeodomain peptide tag operably linked to a therapeutic agent or
(b) a viral vector comprising a nucleic acid sequence encoding a homeodomain peptide tag operably linked to a therapeutic agent,
wherein the homeodomain peptide tag is 15-35 amino acids in length, has at least 80% identity to SEQ ID NO:5 or SEQ ID NO:6, has cellular penetration and secretion functions, and facilitates blood-brain barrier transport of a therapeutic agent in the mammal.

2. The method of claim 1, wherein the homeodomain peptide tag has at least 95% identity to SEQ ID NO:5 or SEQ ID NO:6.

3. The method of claim 1, wherein the homeodomain peptide tag has 100% identity to SEQ ID NO:5 or SEQ ID NO:6.

4. The method of claim 1, wherein the therapeutic agent is an antibody fragment.

5. The method of claim 4, wherein the antibody fragment is D5 or 10H.

6. The method of claim 1, further comprising a linker positioned between the homeodomain peptide tag and the therapeutic agent.

7-13. (canceled)

14. The method of claim 1, wherein a viral vector is administered, and the viral vector is a lentiviral vector.

15. (canceled)

16. A fusion protein comprising a homeodomain peptide tag that facilitates transport of a therapeutic antibody fragment across a blood-brain barrier operably linked to a therapeutic antibody fragment, wherein the homeodomain peptide tag is 15-35 amino acids in length, has at least 80% identity to SEQ ID NO:5 or SEQ ID NO:6, and has cellular penetration and secretion functions.

17. The fusion protein of claim 16, wherein the homeodomain peptide tag has at least 95% identity to SEQ ID NO:5 or SEQ ID NO:6.

18. The fusion protein of claim 16, wherein the homeodomain peptide tag has 100% identity to SEQ ID NO:5 or SEQ ID NO:6.

19. The fusion protein of claim 16, wherein the therapeutic antibody fragment is D5 or 10H.

20. The fusion protein of claim 16, further comprising a linker positioned between the homeodomain peptide tag and the therapeutic antibody fragment.

21. A viral vector comprising a nucleic acid sequence encoding a homeodomain peptide tag operably linked to a therapeutic agent.

22. The viral vector of claim 21, wherein the homeodomain peptide tag is 15-35 amino acids in length, has at least 80% identity to SEQ ID NO:5 or SEQ ID NO:6, and has cellular penetration and secretion functions.

23. The viral vector of claim 21, wherein the homeodomain peptide tag has at least 95% identity to SEQ ID NO:5 or SEQ ID NO:6.

24. The viral vector of claim 21, wherein the homeodomain peptide tag has 100% identity to SEQ ID NO:5 or SEQ ID NO:6.

25. The viral vector of claim 21, wherein the therapeutic agent is an antibody fragment.

26. The viral vector of claim 25, wherein the therapeutic antibody fragment is D5 or 10H.

27. The viral vector of claim 21, wherein a linker is positioned between the homeodomain peptide tag and the therapeutic agent.

28. The viral vector of claim 21, wherein the viral vector is a lentiviral vector.

29. A cell comprising the viral vector of claim 21.

30. (canceled)

31. The cell of claim 29, wherein the cell is a mammalian cell.

32-34. (canceled)


SEQUENCE LISTING (8 sequences)

SEQ ID NO: 1 - DNA, 81 bp, Artificial Sequence
  Description of Artificial Sequence Synthetic oligonucleotide
cagagcctggcccaggaactgggcctgaacgaacgacagatcaaaatctggttccagaaccgccgcatgaagtggaaaaaa

SEQ ID NO: 2 - DNA, 87 bp, Artificial Sequence
  Description of Artificial Sequence Synthetic oligonucleotide
atgggacagagcctggcccaggaactgggcctgaacgaacgacagatcaaaatctggttccagaaccgccgcatgaagtggaaaaaa

SEQ ID NO: 3 - DNA, 48 bp, Artificial Sequence
  Description of Artificial Sequence Synthetic oligonucleotide
cgacagatcaaaatctggttccagaaccgccgcatgaagtggaaaaaa

SEQ ID NO: 4 - DNA, 33 bp, Artificial Sequence
  Description of Artificial Sequence Synthetic oligonucleotide
cagagcctggcccaggaactgggcctgaacgaa

SEQ ID NO: 5 - protein, 27 aa, Artificial Sequence
  Description of Artificial Sequence Synthetic peptide
QSLAQELGLNERQIKIWFQNRRMKWKK

SEQ ID NO: 6 - protein, 29 aa, Artificial Sequence
  Description of Artificial Sequence Synthetic peptide
MGQSLAQELGLNERQIKIWFQNRRMKWKK

SEQ ID NO: 7 - protein, 16 aa, Artificial Sequence
  Description of Artificial Sequence Synthetic peptide
RQIKIWFQNRRMKWKK

SEQ ID NO: 8 - protein, 11 aa, Artificial Sequence
  Description of Artificial Sequence Synthetic peptide
QSLAQELGLNE